(19) United States
(12) Patent Application Publication
(10) Pub. No.: US 2017/0253848 A1
(43) Pub. Date: Sep. 7, 2017

(54) METHOD, SYSTEM AND CONTROLLER FOR PROCESS CONTROL IN A BIOREACTOR

(71) Applicant: Stratophase Limited, Hampshire (GB)

(72) Inventors: Gregory Daniel Emmerson, Winchester, Hants (GB); Samuel Paul Watts, Winchester, Hants (GB); George Edward Barringer, JR., Groton, MA (US); Peter George Robin Smith, Romsey, Hants (GB)

(21) Appl. No.: 15/511,370

(22) PCT Filed: Sep. 2, 2015
(86) PCT No.: PCT/EP2015/070025

§ 371 Date: Mar. 15, 2017

(30) Foreign Application Priority Data
Sep. 15, 2014 (GB) 1416233.3

(51) Int. Cl. C12M 1/36; C12Q 3/00

(52) U.S. Cl. CPC C12M 41/48; C12Q 3/00

(57) ABSTRACT

A method of controlling additive delivery during a bioreaction in a bioreactor comprises running a bioreaction in a bioreactor including adding additive into the bioreactor during spaced-apart feed events, where contents of the bioreactor equilibrate during a stabilisation period after a feed event; making in situ measurements of a bulk physical property of the bioreactor contents during the bioreaction to obtain process trend data; calculating a derivative of process trend data obtained over a measurement period beginning after a stabilisation period, the derivative being a metabolic rate index (MRI); and using the MRI to determine a time for starting a next feed event. A controller for a bioreactor and a bioreactor system are configured to operate according to the method.

### BACKGROUND OF THE INVENTION

The present invention relates to systems and methods for automatic control of processes within bioreactors, for example feeding processes.

In current industrial practice, bioreactors are widely used for producing biological products. Typically the bioreactions which are managed in these bioreactors involve one or more nutrients or feedstocks. Some of these, such as glucose and glutamate, are chemically well-defined molecules, while others, such as fetal bovine serum, are more complex feedstuffs. The nutrients are essential for healthy growth of the organisms of interest, leading to the production of high volumes of good quality titre. In simple batch bioreactor processes all of the nutrients are introduced at the start of the process, which then runs to an end point when most of the nutrients have been consumed or the process self-terminates. An example of such a process is the brewing of wine or beer. However, in more sophisticated bioreactor processes it is advantageous to employ repeated addition of nutrients as this enables extended production and keeps the cells healthy for longer.

Feeding regimes can follow a variety of programmes. A bolus-fed regime uses a pellet or defined volume of nutrients (normally dissolved in water) which is added on a regular, often daily, basis. A continuous regime employs a liquid nutrient which is continuously fed into the bioreactor. Perfusion is a technique in which a feeding regime is employed is conjunction with product removal.

A feeding regime can be developed through experimental optimization to be a pre-defined protocol of feeds (rate or frequency of feeding, volume of feed, etc.) which are applied to the bioreactor. However, it is known to make measurements on samples taken from the bioreactor to assess the requirement for further feeding and possibly adapt the feeding protocol accordingly. Such measurements are often made by sampling a small volume of fluid, which is then taken to an off-line measurement apparatus for chemical analysis. Based on the results of these measurements the feed protocol can be modified at the discretion of a skilled operator. However, extraction of the sample may contaminate the bioreactor contents, so this approach is risky.

Systems are known which can measure a specific single chemical parameter in a bioreactor, for example pH or glucose level, and this can be used for feedback control of the feeding protocol. However, the measurement reflects just one parameter which may have only an indirect bearing on the desired operating regime, since useful production of a complex cellular product may actually depend on many factors including concentration of multiple components, past history, cell density and others. Also, measurement of a single chemical parameter may have little meaning for a bioreaction using a complex feedstuff such as fetal bovine serum.

An example known technique obtains the viable cell count (VCC) within a bioreactor; this is inferred from capacitance measurements made both on-line and off-line. The VCC values are used together with previously determined models about the reaction (stoichiometric relationship between cellular activity and glucose consumption) to estimate current glucose consumption from which a next requirement for feeding can be inferred [1]. Sampling of the bioreactor contents or deconvolution of the inductance measurements to derive the VCC are both necessary, however.

A second example is a feedback-based sampling process with frequent off-line sampling for determining the absolute composition of the reactor contents; this is used as part of the feedback to determine the next feeding time or volume [1]. This process is effective but requires substantial infrastructure to operate. The control may be simplified somewhat by using previously calculated ratios of required feed components (determined empirically from previous experiments) and using the absolute value as an indicator for the whole composition. This also relies on sampling and model creation for reliable control, however.

A third example technique using probing control operates in a system in which oxygen is fed into a bioreactor. Oxygen uptake is measured as an indicator of cellular activity, and through variation in the feed rate, it is possible to observe the maximum oxygen uptake rate and therefore the glucose consumption relative to a theoretical maximum value [2, 3]. Control of feeding is thereby limited to a consideration of the respiratory state of the cells only, but this information may not reflect the complete condition of the reaction with multiple processes and phases.

Hence, there is a need for improved techniques to control feeding and other processes in bioreactors.

### SUMMARY OF THE INVENTION

Accordingly, a first aspect of the present invention is directed to a method of controlling additive delivery during a bioreaction in a bioreactor, the method comprising: running a bioreaction in a bioreactor including adding additive into the bioreactor during spaced-apart feed events, where contents of the bioreactor equilibrate during a stabilisation period after a feed event; making in situ measurements of a bulk physical property of the bioreactor contents during the bioreaction to obtain process trend data; calculating a derivative of process trend data obtained over a measurement period beginning after a stabilisation period, the derivative being a metabolic rate index (MRI); and using the MRI to determine a time for starting a next feed event.

In some embodiments, the process trend data obtained via the in situ measurements is actual process trend data, and the method further comprises applying a mathematical method to actual process trend data obtained over a measurement period beginning after a stabilisation period to produce calculated process trend data representing a damped version of the actual process trend data, and the calculating a derivative comprises calculating a derivative of the calculated process trend data. The mathematical method may comprise an averaging of the actual process trend data, or a fitting of a mathematical curve such as a second order polynomial curve to the actual process trend data. Other mathematical methods and curves may also be used.

The bulk physical property may be refractive index. In this case, making in situ measurements of refractive index may comprise using a sensor configured to detect changes in a propagating evanescent wave, such as a sensor the detects changes in a modal index, and/or a sensor which is based on a Bragg grating.

Alternatively, the bulk physical property may be density, conductivity, inductance, impedance, viscosity, turbidity, or spectral absorption at a single wavelength.

The method may comprise using the MRI directly to determine a time for starting the next feed event. The MRI may alternatively be used indirectly. For example, the method may comprise using the MRI to determine a time for starting the next feed event comprises calculating a ratio by dividing the instantaneous MRI value by an absolute maximum value of the MRI since the previous feed event, comparing the ratio to a threshold value, and starting the next feed event when the ratio passes the threshold value. In some embodiments, the threshold value may be in the range of 0.3 to 0.9.

The measurement period may commence with a minimum temporal window, with the applying of the mathematical method and calculating of the derivative beginning on process trend data collected during the minimum temporal window after the minimum temporal window expires. This can improve accuracy of the mathematical method and the MRI. In this regard, the minimum temporal window may have a duration such that sufficient process trend data is collected for the corresponding MRI to have a minimum error or noise value compared to that for other durations. Further, the duration of the minimum temporal window may be adjusted based on previous process trend data.

The stabilisation period may have a duration determined by observation of process trend data obtained during a calibration run of the bioreactor. However, other and automated techniques for setting the stabilisation period are not precluded.

The method may further comprise applying noise filtering to the obtained process trend data, to improve the quality of the measurements. There can be many sources of signal noise in a bioreactor system and a sensor, so it is desirable to improve the signal-to-noise ratio when possible.

The method may also comprise varying the amount of additive delivered in a feed event in response to the MRI. In this way, both the feed times and quantities are adjusted in response to the measurements, providing detailed control of the reaction. In some embodiments, no additive is added into the bioreactor between feed events, and in other embodiments, additive is added into the bioreactor at a first rate during each feed event, and additive is added continuously into the bioreactor between feed events at a second rate lower than the first rate. One or both of the first rate and the second rate may be varied over time.

Furthermore, different additives may be added into the bioreactor during different feed events, the MRI during a current measurement time being used to determine the additive for the next feed event. This enables automated control of more complex bioreactions.

In any embodiment, the or each additive is a direct or indirect feedstock. Other additives may also be employed, according to the nature of the bioreaction.

The method may further comprise using the MRI to determine values for one or more operating conditions of the bioreactor, such as temperature. This offers more sophisticated control, possibly complete automated control of many aspects or every aspect of the bioreactor.

A second aspect of the invention is directed to a controller for controlling additive delivery during a bioreaction in a bioreactor, the control configured to: receive in situ measurements of a bulk physical property of contents of a bioreactor during a bioreaction, the bioreaction including additive being delivered into the bioreactor during spaced-apart feed events, where contents of the bioreactor equilibrate during a stabilisation period after a feed event, the received measurements being process trend data; calculate a derivative of process trend data obtained over a measurement period beginning after a stabilisation period, the derivative being a metabolic rate index (MRI); use the MRI to determine a time for starting a next feed event; and generate a control signal configured to cause an additive supply mechanism to deliver additive into the bioreactor at the determined time for the next feed event.

A third aspect of the invention is directed to a system for controlling additive delivery during a bioreaction in a bioreactor, the system comprising: a bioreactor, an additive supply mechanism configured to deliver additive into the bioreactor during feed events, in response to a control signal; a sensor associated with the bioreactor and configured to make in situ measurements of a bulk physical property of the bioreactor contents during a bioreaction, thereby obtaining process trend data; and a controller configured to: receive process trend data from the sensor; calculate a derivative of process trend data obtained over a measurement period beginning after a stabilisation period during which contents of the bioreactor equilibrate after a feed event, the derivative being a metabolic rate index MRI; use the MRI to determine a time for starting a next feed event; and send a control signal to the additive supply mechanism to deliver additive at the determined time for the next feed event.

### BRIEF DESCRIPTION OF THE DRAWINGS

For a better understanding of the invention and to show how the same may be carried into effect reference is now made by way of example to the accompanying drawings in which:

FIG. 1 shows a schematic representation of a first example bioreactor system that can be controlled according to embodiments of the invention;

FIG. 2 shows a schematic representation of a second example bioreactor system, having multiple feedstocks, that can be controlled according to embodiments of the invention;

FIG. 3 shows a schematic cross-sectional view of a first example bioreactor suitable for use in the systems of FIGS. 1 and 2;

FIG. 4 shows a schematic cross-sectional view of a second example bioreactor suitable for use in the systems of FIGS. 1 and 2;

FIG. 5 shows a graph of an idealised in situ measurement of a non-process specific parameter over the duration of two feed cycles in a first example bioreaction in a bioreactor that can be controlled according to embodiments of the invention;

FIG. 6 shows a graph of an idealised in situ measurement of a non-process specific parameter over the duration of two feed cycles in a second example bioreaction in a bioreactor;

FIG. 7 shows a graph of experimental in situ measurements of refractive index over the duration of a feed cycle in an actual bioreaction;

FIG. 8 shows the data of FIG. 7 together with a fitted polynomial curve (calculated process trend).

FIG. 9 shows a graph of process trend data for four feed cycles of a real bioreaction, together with calculated derivative curves (metabolic rate index, or MRI);

FIG. 10 shows a graph of the MRI curves from FIG. 9 together with trigger data derived from the MRI curves that can be used to control feed events according to embodiments of the invention;

FIG. 11 shows a graph of an idealised in situ measurement of a non-process specific parameter over a feed cycle in a bioreaction have feed delivered at a non-zero background level and controlled in accordance with embodiments of the invention;

FIG. 12 shows a graph of MRI and local feed consumption rate in a bioreaction having varying non-zero background and peak feeding levels controlled in accordance with embodiments of the invention; and

FIG. 13 shows idealised MRI values over multiple feed cycles in a bioreaction fed from multiple feedstocks controlled according to embodiments of the invention.

### DETAILED DESCRIPTION

The present invention addresses the drawbacks with existing bioreactor process control techniques by using particular mathematical processing of in situ measurements of a non-specific process parameter to derive a feeding protocol which is then delivered automatically. The protocol is adjusted throughout the process lifetime in response to the measurements, so that control of the bioreaction is automated from start to completion.

By “non-specific process parameter” is meant a bulk physical property or parameter of the contents of the bioreactor during the bioreaction, typically the liquid contents, but also or instead the gaseous content that occupies the bioreactor volume above the liquid, known as the “head space”. Such properties have an instantaneous single value that varies over time with compositional changes in the contents, for example when feedstock is added and as the feedstock is consumed. However, these parameters are such that they cannot be deconvolved down to a measurement of any specific compositional component (such as the amount of glucose), but are instead functions of the overall composition of the bioreactor contents at that moment. In other words, they are not specific to any bioreaction component; they are “non-specific”, and do not represent the value of any individual component of the bioreaction composition. Parameters of this type include refractive index, density, conductivity, inductance, impedance, viscosity, turbidity, and spectral absorption at a particular wavelength; these parameters are such that their value is not dominated by or correlated to a single compositional component in the bioreactor. Since the parameters cannot reveal any information about the actual composition of the bioreactor contents as regards the individual components making up the contents, it is surprising that they can be of use in controlling the process to which that contents is subject. Nevertheless, the present inventors have found that the variation of these parameters reflects changes in the overall contents composition arising from the addition and consumption of feedstocks and other materials and the growth and decline of the cell population. Hence, monitoring the rate of change of one or more such parameters enables the deduction of protocols for controlling the process, such as when to add feedstocks and other additives.

The invention is not limited to controlling feedstock addition; it is also applicable to other nutrients or additives which may be used to promote a particular pathway or set of reactions, either in the primary organisms or in secondary reactions. An example of this is the addition of enzymes into a mixture that has had glutamate added, to produce glutamine for use by the desired cells. In this case, glutamate would likely be considered a feedstock, while the glutamine synthetase (enzyme) might not be considered a feedstock because its role is in transforming the glutamate feedstock into useful glutamine. A further example is the addition of gaseous ammonia from which nitrogen is then derived for use by the cells. The invention can be used to control the addition of additives of this type since the measured bulk physical parameter responds also to the effect of the added component on the product rate. By controlling via such a non-specific measurement the invention enables, in the first example, control of the addition of both the glutamate (the feedstock—albeit an indirect feedstock) and the enzymatic enabler to promote the efficiency of the overall system. Hence, in the present application, the term “additive” is used for any component, material or ingredient, gas, liquid or solid, which is added to the bioreactor during the bioreaction, including direct feedstocks, indirect feedstocks, converters or transformers such as enzymatic enablers that produce usable feedstocks from other ingredients, and any other component required for the bioreaction to be maintained. Addition or injection of an additive into the bioreactor is termed a “feed event” (even if the additive is not a direct feedstock material), and takes place over a finite time.

According to the invention, measurements of a selected non-specific process parameter are made in situ, that is, directly inside the bioreactor on the contents, without the extraction of samples. In situ measurements are of particular benefit for monitoring and controlling bioreactions, because the reactions are so vulnerable to contamination that can occur during sample extraction. Therefore, the present invention employs one or more sensors or probes configured for bulk parameter measurement and which can be deployed inside a bioreactor vessel and left in place at least for the duration of the process during which it is in contact with the contents. Advantageously, the sensor is configured to remain in place during sterilisation of the reactor, and is not damaged or degraded thereby. Hence, the choice of probe is important; it is desirable for the probe to be inserted into the reactor vessel at a point representative of the bulk volume process, and that it is compatible with any sterilization or cleaning processing required for the bioreactor. For re-usable reactor vessels which are typically made from metal or glass the sterilization is often carried out by the use of autoclave or “steam-in-place” which provides a hostile environment for some types of sensor causing failures and device degradation. An alternative to the sterilization of a re-usable reactor is the use of single-use bioreactors (which may be plastic, for example) which are normally gamma-irradiated for sterilization and in which sensors are installed at manufacture.

The sensor comprises one or more transducer elements for transducing the changing physical parameter into a measurement signal, and a transmitting element for communicating the signal out of the reactor to a processor and control unit that processes the signal and determines the additive protocol therefrom. The transmitting element will likely be an electrical cable or an optical fibre (or bundle or ribbon of optical fibres), depending on the nature of the transducer element, although wireless communication of the signal is not precluded. Various converters may be required to convert between the signal as measured and the signal to be transmitted. Also, the sensor may be configured to perform some or all of the signal processing that is carried out on the measurement signal according to embodiments of the present invention, before transmitting the result to the next part of the system. Hence, part or all of the functions of the processor and the control unit may be incorporated into the sensor device. Division of the signal processing functionality between the sensor and the control unit processor may be configured and implemented in any manner considered convenient.

An advantage of the present invention is its ability to utilize a sensor which is not specific to a particular nutrient or component of the bioreactor media or contents, but rather one which has a response which varies with nutrient and product concentrations, waste, and any other compositional changes, that is, a sensor that measures a bulk physical property. For example, the refractive index of a reactor contents will depend on glucose and glutamate, but will also depend additionally on lactate and protein production, and any other by-product of the process which may or may not be known. By monitoring the response of the bioreactor system using a non-specific measurement such as refractive index, and using knowledge of feed pulses or changes in feed rate, sufficient information can be produced so as to allow for automated control of a bioprocess via a feedback procedure. The details of this control are discussed in more detail later.

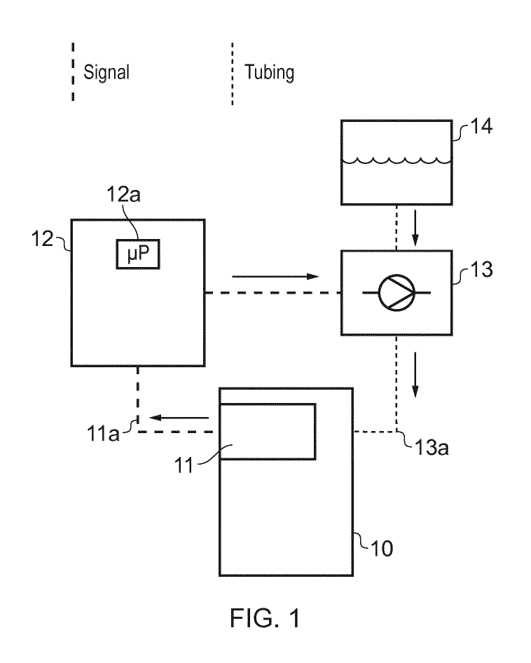

FIG. 1 shows a schematic representation of an example system which can be controlled according to embodiments of the present invention. The various components are connected by tubing for the flow of additives (shown by the dotted lines) and by signal lines for transmission of the signal representing the measured parameter and transmission of control signals to control the process (shown by the dashed lines). A bioreactor 10 may be either single use or reusable, and may be made from any of various materials, including glass, metal and plastics. The volume of the reactor may range from microliter volume in a micro-reactor up to tens of thousands of litres in a large volume industrial production reactor. The invention is applicable regardless of reactor size, so the reactor volume may be any volume in which the required bioprocess can be successfully operated. A sensor 11 is positioned inside the reactor vessel for in situ monitoring of the contents of the bioreactor 10, by measuring the selected bulk physical property. This may be a property of a liquid content (for example, its refractive index), but could include measurements of gas in the head space, or other properties (such as dielectric properties and mass build-up). The measurements are obtained in situ, and are passed by an electrical or optical cable 11a to a controller 12 which receives the sensor measurement signal. The controller 12 includes a microprocessor 12a programmed with software (or any equivalent data processing element or device implemented in hardware, software or firmware) to process the received signal and interpret the resulting data in order to produce control signals. The details of this are discussed further later. The control signal or drive signal, which may be electrical using for example analogue voltage or analogue, using a protocol such as Ethernet, I2C, USB, RS485, OPC, etc., is passed (by cable or wirelessly) to a pump 13 to control the pump's operation. The pump 13 is a pump system or valve which obtains from an additive reservoir 14 a required volume or quantity of additive (feedstock, etc.) and injects this amount of fluid or solid feedstock into the bioreactor via tubing 13a. The additive reservoir may store the additive as a single component, as an aqueous solution such as glucose solution, or as a multicomponent solution or product (for example, containing glucose, glutamate, salts, amino-acids, buffers, and other components as a mixture). Note that the controller 12 and the pump system may be integrated into the same physical enclosure or housing (for example, to form a control-tower). Further, or alternatively, the microprocessor 12a may be disposed within a housing of the sensor 11 such that the sensor 11 is configured to deliver control signals directly to the pump 13 without the need for a separate controller. As a further alternative, the microprocessing functions may be divided between the sensor 11 and the controller 12, with both provided with a microprocessor 12a. Also, the pump 13 and the reservoir 14 may together be considered as an additive supply mechanism. Such a mechanism might be configured in an alternative manner; the requirement for the mechanism is simply that it can automatically deliver a required quantity of additive into the bioreactor vessel in response to a control signal.

Note that the in situ measurements of the bioreactor contents may alternatively be obtained using an external sensor that can obtain measurements of an appropriate non-specific process parameter of the contents within the reactor while not being itself disposed inside the reactor volume. Some sensors are configured for “remote” sensing, where a sensing signal such an optical beam is transmitted through the reactor wall or a window therein. Refractive index, absorption and turpidity may be measured in this way, for example. Hence, in the context of the present invention, the phrase “in situ measurement” is to be understood as meaning that the measurement is made on the bioreactor contents while it is reacting inside the bioreactor, rather than known techniques that rely on extraction of a sample of the contents for testing elsewhere. In other words, the measurement is in situ, but the measurement device may not be in situ in the strictest sense, rather it is associated with, proximate to or in the vicinity of the reactor to measure conditions inside the reactor.

Also, the system may include other components (not shown) such as a temperature sensor, heater controller and heater element configured to provide a feedback loop for temperature control in the bioreactor. Other feedback loops may be provided for control of conditions such as oxygen level, pH, stirring and carbon dioxide level.

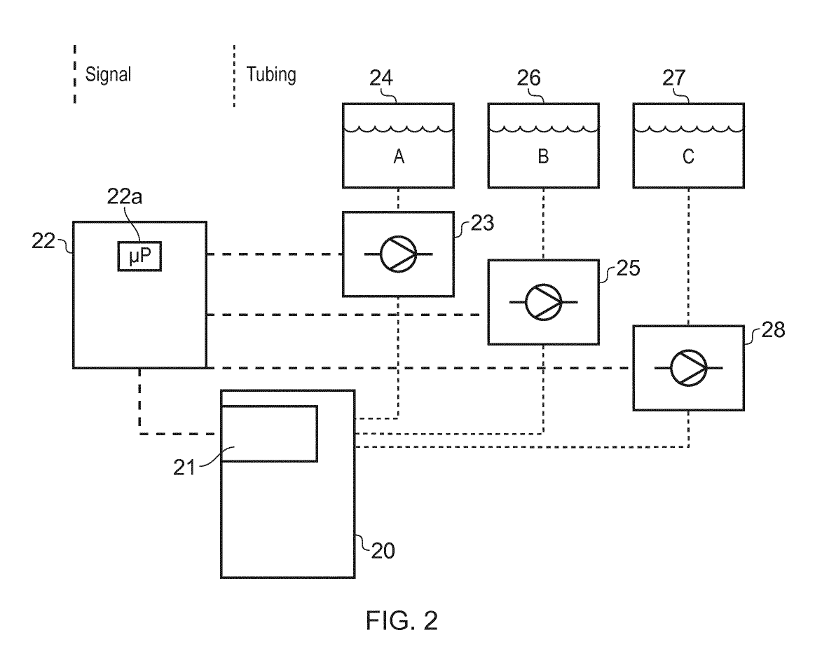

FIG. 2 shows a schematic representation of a further example system that may be controlled according to embodiments of the present invention. This system comprises a bioreactor 20, an in situ sensor 21, and a controller 22 with a processor 22a which are the same as or similar to the equivalent components in FIG. 1. In this example, however, the controller 22 has multiple control signal outputs such that it is able to control a plurality of pumps 23, 25, 28. Each pump is connected to a corresponding separate additive reservoir 24, 26, 27. Additives A, B and C in the reservoirs 24, 26, 27 may be ingredients such as nutrients or feedstocks of entirely different compositions, or having at least some common components but in different ratios or concentrations. More additives may be desired, and the system can be extended accordingly with further pumps and reservoirs, each pump receiving a control signal from the controller 22. As depicted, each pump feeds a separate injection port into the bioreactor 20. Alternatively, a mixing manifold could be used to combine the additive flows from the various pumps into fewer or one injection port. In such an arrangement, it is desirable to prevent backflow into unused additives in the tubing; this can be accomplished by injecting the additives into the gas-filled head-space, and by the use of peristaltic pumps that prevent backflow.

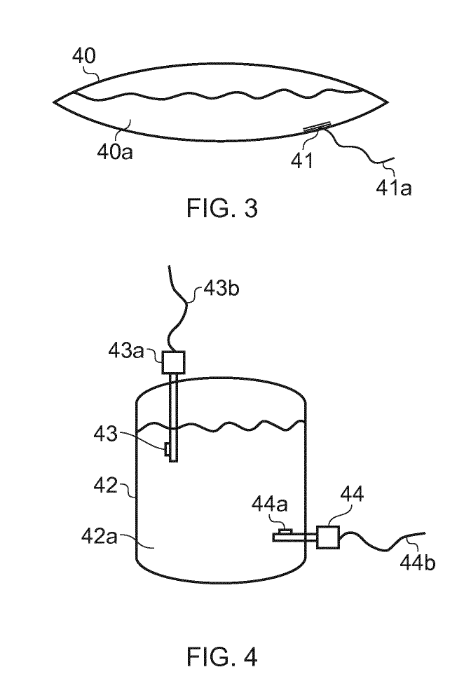

FIG. 3 shows a schematic cross-sectional view of a bioreactor vessel configured for use in a system operated according to embodiments of the invention. The bioreactor 40 is a single use bioreactor in a bag format (typically a non-rigid plastic bag) with a sensor 41 permanently welded, adhered or otherwise affixed into the side wall of the bag such that it is exposed to the liquid contents 40a of the reactor 40. Hence, the sensor 41, which may be in the form of a chip contained in a housing permanently attached to the inside of the bag, is integrated into the structure of the reactor 40. The sensor 41 has a signal line 41a (for example, electrical or optical cable, or free space signal transmitter) for relaying the measurement signal outside of the reactor bag wall; this will be connected directly or indirectly to a controller such as those shown in FIGS. 1 and 2. The sensor 41 is fixed throughout the life cycle of the reactor 40 and provides an in situ monitoring of the contents of the bag, namely the liquid 40a. The sensor 41 will be subjected to the same sterilising irradiation (typically gamma) as the rest of the bioreactor.

FIG. 4 shows a schematic cross-sectional view of an alternative bioreactor vessel configured for use in a system operated according to embodiments of the invention. In this example, the bioreactor 42 operates with an insertion probe that houses the sensor and which is configured for use in a rigid reaction vessel having a through-port into which the probe is inserted in fluid-sealed manner. The reactor 42 is a reusable reactor vessel. These are usually of glass or steel construction and are designed with a lifetime that allows for hundreds of cycles of use. Such reactors have capacities in the range of hundreds of millilitres to tens of thousands of litres. At the point of manufacture the vessels are provided with multi-function ports manufactured into the skin or wall of the vessel which allow probes to be fitted to the reactor, to measure conditions inside. These ports are typically of a generic format (although often of a particular manufacturer's design, for example an Ingold® port for use with various probes that conform to certain design formats (such as a 12 mm or 25 mm diameter shaft). The reactors are designed such that the fully assembled reactor can be steam-sterilized either though placement in an autoclave (for smaller reactors) or steam-in-place (SIP) during which the interior of the reactor is exposed to superheated steam (for larger reactors). In both of these arrangements one or more sensors (housed in probes configured for the ports of the vessel) can be mounted in-reactor during the sterilization cycle. As illustrated, a sensor 43 is mounted in a reusable probe format 43a. The probe 43a may be of a stainless steel construction configured to fit through an existing multi-function port in the reactor vessel 42. The probe 43a then forms part of the boundary of the reactor vessel 42. For benchtop bioreactors (typically <20 L), a probe 43a may be mounted for convenience into a port in the headplate of the bioreactor 42 and hence needs to reach down such that the sensor 43 is immersed into the media to be measured (contents 42a of the bioreactor 42). This will typically require longer format probes of a length greater than 100 mm. As an alternative, a sensor 44 is shown housed in a probe 44a mounted through a port positioned in the side wall of the reactor 42, so that the sensor reaches directly into the contents 42a. It is convenient for larger SIP reactors (>20 L) to have the ports on the side located towards the bottom of the reactor 42. This typically requires a shorter probe length, such as less than 150 mm. In each case the probe 43a, 44a has a signal line 43b, 44b to communicate the measurements from the sensor 43, 44 to the controller.

A further alternative configuration is to provide a sampling or bypass tube in which fluid travels from the reactor vessel to a measurement site having an internal or associated sensor for in situ measurements, and is then recirculated back into the main body of the vessel, or passed to waste.

As mentioned, refractive index is an example of a bulk physical parameter which may usefully be employed in the present invention. Any sensor capable of measuring refractive index, directly or indirectly (i.e. the sensor measures a property from which refractive index can be deduced), in situ in a liquid or gas may be used. Examples of suitable sensors are ones which are based on one or more Bragg gratings. Sensors of this type are described in PCT/GB2005/002680 and PCT/GB2005/002682. A Bragg grating in a planar waveguide has a particular spectral response and is provided with an overlying window for receiving a fluid sample. The presence of a sample in the window affects the effective modal index experienced by the evanescent wave of light propagating in the grating, to modify the grating response. The grating response depends to the refractive index of the fluid so that measurement of the spectral response (by detecting light reflected or transmitted by the grating) gives information about the refractive index. A change in the fluid refractive index, as occurs in the contents of a bioreactor as the reaction occurs, causes a spectral shift in the reflected optical signal, so that monitoring of the reflected optical signal gives an indication of the changing pattern of the refractive index. This optical change can be read out in various different ways, for example by using a broadband optical source to direct light to the grating and a spectrally resolving detector (for example, an optical spectrum analyser) to collect the reflected light, or by having a tunable laser and a power detector, or by utilizing one of many known commercial approaches such as a grating interrogator based upon tunable filters. The raw signal from such a sensor can be averaged and filtered to reduce the effect of random noise and give improved precision to the recorded signal that itself is a function of the refractive index of the fluid being measured.

Commercially available Bragg grating-based refractive index sensors that operate in this manner are produced by Stratophase Limited, as the Ranger Probe device (http://www.stratophase.com/downloads/Ranger-Probe-Technical-Specification-V2.1. pdf).

Other refractive index sensors may be used, however, such as sensors configured to detect evanescent wave changes and model index in a different manner (e.g. Mach Zehnder-based sensors), or sensors configured to measure any other non-specific process parameter as defined above. Measurements of any of the parameters with any suitable type of sensor can be handled in the same way to derive control signals in accordance with the invention.

Whichever non-specific process parameter is chosen, it will vary over time as the biological process in the bioreactor proceeds. When feedstock or other additives are introduced into the bioreactor, a significant change in the composition of the bioreactor contents takes place; this is reflected in a changing value of the process parameter. By monitoring this changing value, the present invention provides a way to determine when it is appropriate to inject the next dose of additive. This can continue over many additive cycles until the process reaches a desired end point, and is able to offer improved process outputs compared to a regular feeding regime in which additives are injected at pre-determined regular intervals, without reference to the contents' composition.

Changes in the bioreactor contents composition are not merely due to changing levels of feedstock as it is added and consumed. Bioreactions are complex, and the non-specific process parameter, for example refractive index, will change due to both feedstuff level and cellular metabolisation (and possibly other factors). Consequently, it is not a simple matter to deconvolve the underlying concentration of specific components such as glucose from a measurement of the chosen bulk process parameter. According to the invention, a different approach is taken, in which a non-specific process parameter signal (for example the refractive index trend) is recorded and used to determine information about the reactor contents with no attempt to derive values for specific compositional data.

When a bulk property non-specific process parameter is monitored, the present inventors have recognised that a single additive cycle (the time from injection of an additive dose to the time when a next dose is required) can be understood as being made up of three distinct phases. If the total duration of the cycle is designated as t4, the cycle can be described as t4=t1+t2+t3.

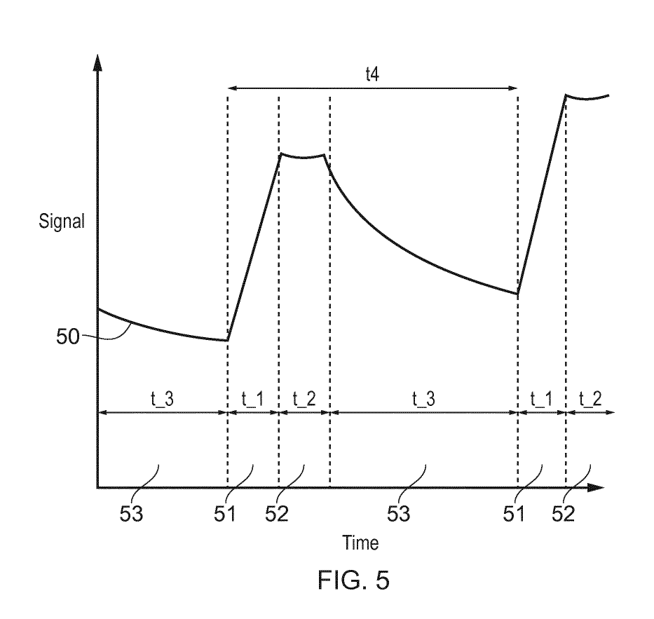

FIG. 5 shows a plot of an idealised temporal response of a in situ measured non-specific process parameter (the signal) to a feed event. The temporal response, for example refractive index variation, also described herein as the process trend (since it describes the trend in the bioreaction process) is shown as line 50 on a plot of signal level against time. The cycle length t4 begins with a time period t1, shown as 51. This is the duration of a feed event or feed pulse, when a dose of feedstock or other additive is injected into the bioreactor. The injection takes a finite time t1, and during this time the change in the signal 50 is dominated by the addition of the feedstock. In this example there is a large increase in signal response to the added material. This will happen, for example, if glucose is added to an ethanol production process.

At the end of the feed event (end of t1), the next phase t2 begins, shown as 52. This is a transient stabilization stage during which chemical mixing and transient biological and metabolic processes equilibrate. The duration of t2 is discussed further below.

At the end of the stabilisation stage, the next phase t3 begins, shown as 53. This phase is a measurement stage, during which the feed added during the feed event is consumed, and the changing measured signal value is used to determine when to begin the next feed event, in accordance with the invention. This is discussed further below. Once the next feed event start time is determined, and that time has arrived, the measurement stage t3 stops, the cycle t4 is complete, and the next feed event begins, taking time t1. Hence, the cycle repeats t1, t2 and t3 as required, until the bioreaction process is complete.

In this example, during the measurement stage t3 the signal is dropping because, for example, the glucose concentration is reducing as it is consumed, and ethanol is produced in consequence. Ethanol has a similar refractive index to the underlying aqueous environment of the bioreactor contents, and so there is a net drop in refractive index. Hence, if one is measuring refractive index, a signal 50 of the form shown in FIG. 5 would be expected.

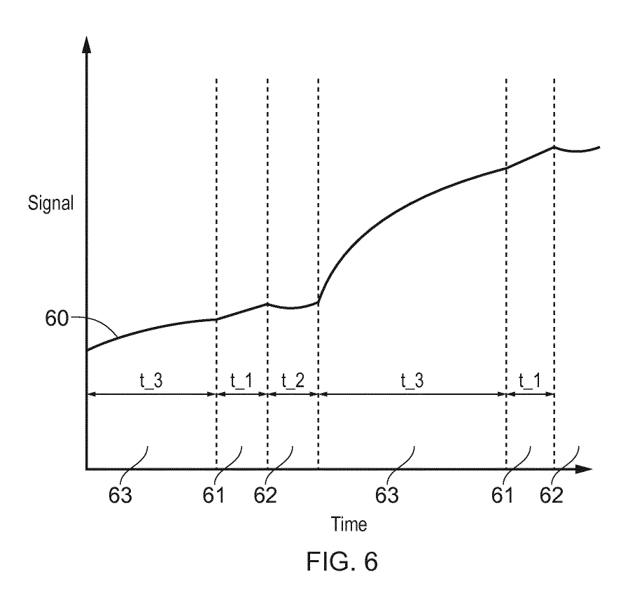

FIG. 6 shows a plot of a second idealised temporal response of a in situ measured non-specific process parameter (the signal 60) to a feed event, but for a different type of bioreaction. Addition of feed during the feed event t1 (61) produces a relatively small change in the signal 60 which in this example is measured refractive index. This would typically occur if the bioreaction product has a greater effect on the refractive index than the feed-stuff, an example of which could be addition of glucose to a protein production in CHO mammalian cells where the protein has a much larger effect on refractive index. Hence the measured signal is dominated by the amount of product, and not by the feedstock as it is added and consumed. Hence, the signal 60 increases only a small amount when the feedstock is added

After the feed event ends the stabilisation stage t2 (62) begins, and leads to the measurement stage t3 (63), as before. In this example, the t3 phase sees an increase in signal 60 (refractive index) as proteins are produced in response to the consumption of the feedstock. Note that the underlying nutrients (feedstock) are being depleted so that the contribution of the glucose concentration to refractive index is dropping too, but the increase in protein and other by-products more than offsets this negative contribution and so the overall trend is an increase in signal during t3. The measurement stage t3 terminates when the next feed event is scheduled in response to the measurements made during t3.

Contrast this process overall, where the addition of feedstock produces a small positive change in signal and the consumption of feedstock produces a large positive change in signal, with the process of FIG. 5, where the addition of feedstock produces a large positive change in signal and the consumption of feedstock produces a large negative change in signal. Other patterns are also possible, for example a process using methanol as a nutrient source could produce a reduction in refractive index due to dilution effects, so that the signal would show a negative change during t1. The variation of the signal over the cycle time t4 depends entirely on the nature of the bioreaction, the additives and the product, but the concept of dividing the cycle time t4 into the stages t1, t2 and t3 still holds, and the control method according to embodiments of the invention is applicable regardless of signal change direction at each stage.

The stabilisation stage, t2, is the time between the end of the feed event and the start of the measurement stage. The measurement stage t3 is a time period during which the signal is used to determine the next feed event start time, and it is preferable that the signal is behaving in a particular manner when this determination is made, so that meaningful results are obtained. The bioreaction will undergo a period of stabilisation after a feed event before the behaviour appropriate for the measurement period begins, and the length of this stabilisation period should be determined so that t2 can be set. In some embodiments the duration of t2 is kept the same for every cycle t4, while in other embodiments, t2 can be dynamically adjusted in response to current or recent measurements, for example to cope with changing component volume within the reactor. To determine the length of t2, a possible procedure is to conduct an initial calibration run in the bioreactor which is representative of the bioreaction, feedstocks and cell type which will be involved, and to observe the resulting nonspecific process parameter signal measured with the in situ measurement sensor over least one additive cycle (for example, the refractive index trend curve). By observation of the transient reaction associated with and following a feed event (the small dip in the measurement signal shown in FIGS. 5 and 6, for example), one can choose a duration for t2 such that transients are reduced or finished and the measurement trend is observed to begin to follow the required behaviour (which can be determined mathematically and is discussed further below). This time period will vary according to cell type, reaction type and reactor type, so is conveniently determined by observation of a calibration run in this manner. For example, in a mammalian cell process which may last for up to several weeks, the time to recover to equilibrium or stable behaviour post-feed (t2) may be in the range from 15 minutes to 2 hours, and feed events may occur approximately daily (as an example). In contrast, for a microbial process such as E-coli, the total process may run for several hours only with feed events approximately every 5 minutes, so that t2 might fall in the range from 5 seconds to 30 seconds. Optional techniques for setting t2 are discussed further below.

Once t2 has been set, and has elapsed during a cycle time, the measurement stage t3 begins. Mathematical curve fitting and further processing are performed on the signal measured during t3 to determine when the next additive stage t1 (feed event) should begin; this is discussed in more detail later. In order to improve the result obtained from this, it is possible to filter the measured signal to remove noise. During and after any feed event there will be a limited signal-to-noise ratio, and multiple sources of transient events that disrupt the signal, including mixing of the contents as the additive is injected, sparge conditions, and temperature fluctuations (due to internal heat generation or active heating). While some of these events might occur at known frequencies and thus be relatively simple to remove by filtering (for example using Fourier-based approaches), in practice it is found that multiple contributions interact to create noise and it is advantageous to use more robust methods which are tailored to the application, and in particular prove useful with non-specific process parameter sensors. An example of a non-periodic noise contribution comes from small bubbles produced in sparging processes which attach and detach from the sensor surface in a random way causing unpredictable signal variation (noise).

In describing the signal-to-noise ratio it is helpful to recognize that there is noise inherent to any measurement, and various techniques may be implemented to alleviate this noise source. For example, if refractive index is the non-specific parameter of choice and it is measured with a Bragg grating-based sensor as described above, the appropriate choice of noise filtering will depend upon the technique used to record the spectral measurements, but in general will benefit from both repeated spectral measurements during each measurement cycle with curve fitting of the grating position, and also from repeat measurements of the curve fitting over time. Approaches such as moving average, and other more complex filtering may be employed. Other noise reduction techniques may also be employed, having regard to the parameter being measured and the nature of the sensor.

There are, however, further noise-like features (or variability in signal) which are genuine, in the sense that they do represent changes in the reactor contents (and hence in the measured parameter, such as refractive index), and are driven by either physical causes (stirring, temperature changes, etc.), or transient changes in the biological media (for example the response of cells to changing concentrations in the background fluid). These sources of noise, which are genuine process variations, cannot be simply reduced through signal processing (filtering), but must be dealt with in the context of the biological process.

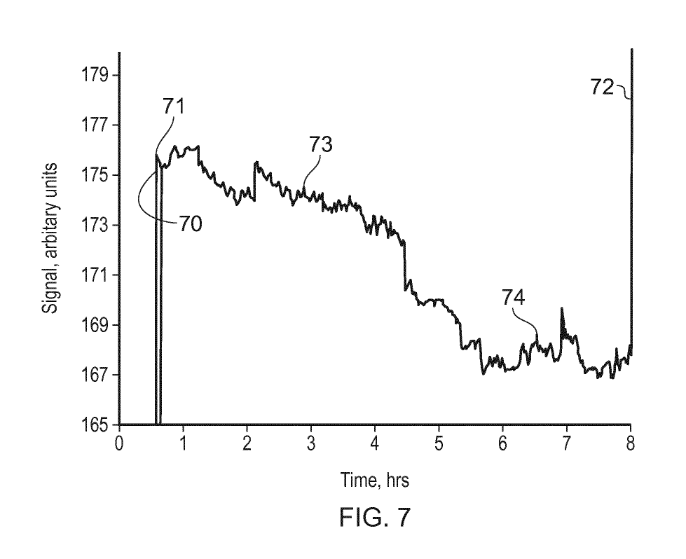

FIG. 7 shows a graph of real experimental data, and illustrates typical noise and variation on the measured trace. The graph shows as a measured signal 70 the spectral shift of a Bragg grating sensor (representing a measurement of refractive index of the bioreactor contents) in normalized units as it varies over time. The bioreactor process was a yeast-based (Saccharomyces cerevisiae) fermentation run at low temperature with glucose in water as the nutrients, and producing ethanol and biomass as its predominant products. This data shows genuine process variation (after appropriate noise filtering has been applied). The nearly vertical lines 71, 72 in the signal 70 occurring at 0.5 hrs and 8 hrs are feed events. The process is feed-dominated by the glucose, so the feed events give a significant positive step in the measured reflected wavelength from the planar Bragg grating sensor. The smaller step feature 73 is a reversible artefact in the trace that can typically be associated with temporary alterations in the sensing region (such as a carbon dioxide bubble) and the later small peak features 74 are typically associated with the flux of biological product in the reactor.

Appropriate filtering and signal processing on the raw signal data as discussed yields data representing the actual trend of the bioreaction process (such as the example of FIG. 7), that is, the highest temporal frequency process trend data, which might be one data value every 2.5 seconds, for example. To then deal with genuine process variation so as to produce data genuinely useful for determining a feed event time, one may
Begin the analysis of the filtered data after the end of t2 (the stabilisation period or blanking during which equilibration is anticipated to have occurred).
Define a minimum temporal window. The length of the window will depend on the process, and may be defined as a time duration, for example 60 seconds, or as a number of collected measurement values or data point, for example approximately 30 to 40 data points. The value for the window may be related back to the data averaging and smoothing performed on the raw spectral data which was described earlier in the context of Bragg grating sensors for measuring refractive index. The minimum temporal window may be thought of as a buffer during which measurement values are accumulated, and may be as long as many hours for very slow processes. The purpose of the minimum temporal window is to enable the next step, which is a mathematical curve fitting step, to be accurate; a minimum amount of data will be necessary to achieve a fit which properly represents the varying signal.
Record measurement values of the signal throughout the minimum temporal window; this is the actual process trend data.
At the end of the minimum temporal window, apply a curve fitting approach to the collected measurements to fit a polynomial curve to the actual process trend data. Any curve fitting approach may be employed, such as least squares, absolute deviation, or non-convex approach. Preferably the polynomial is a low order polynomial. In one embodiment, a second order polynomial (quadratic equation) is used. The measurement collection and curve fitting may carry on after the end of the minimum temporal window. The inventors have determined that polynomial curves, particularly low order polynomials, provide a good model of the variation of bulk property parameters in a bioreactor in a time period after the contents have stabilised following a feed event.
Record an interpolated value from the polynomial (the polynomial coefficients), as representing the now filtered and calculated process trend. It may be beneficial to not make use of the values calculated at the extreme edge of the minimum temporal window, perhaps dropping about five values at the start of the window.
Use the resulting calculated process trend to determine a future feed event, as will be described later.

The length of the minimum temporal window may be set by the designer of the system or the operator of the controlled process. Alternatively, minimum temporal window length may be optimised and re-set by the controller using an automated process calculated from one or more previous complete t4 data cycles. Setting too short a minimum temporal window will result in unwanted oscillations in the calculated process trend (which then makes it difficult for the controller to accurately determine feed events). On the other hand, setting too long a minimum temporal window results in a significant time lag that degrades the performance of the feedback feeding system and loss of significant information and control. Thus, the minimum temporal window is preferably balanced between these extremes. Routes to determine the minimum temporal window may make use of analysis based on sign cross-overs between the low order polynomial fit and the process trend data. Analysis of this information and its dependence on the minimum temporal window length can be used to achieve optimal operation. The exact length of the minimum temporal window is not critical, however.

Returning briefly to the setting of the length of the stabilisation period t2, an appropriate end time for t2 may be determined from the observation of the calibration measurements, noting the approximate point at which the signal begins to follow a curve that can be modelled by the polynomial. Indeed, using the calibration data, one can work backwards by fitting the low-order polynomial to the process trend data, and identifying the junction between t2 and t3 as the point behind which the polynomial ceases to fit. A threshold of deviation from the polynomial fit could be determined, for example, and the time at which the signal exceeds this threshold could be designated as the end point of the t2 period. For example, the end point might be determined as the point in t2 closest to the end of t1 where the local error for the fitted polynomial exceeds two times the average error for the fit over the minimum temporal window (or other time period at the beginning of t3). Otherwise, the end point can be determined as the point where the raw measurement data ceases to intercept with the fitted data (subject to and/or taking account of noise fluctuations in the raw data). Alternatively, a judgement of the end point can simply be made by eye. Other methods for setting the duration of t2 may also be used as convenient.

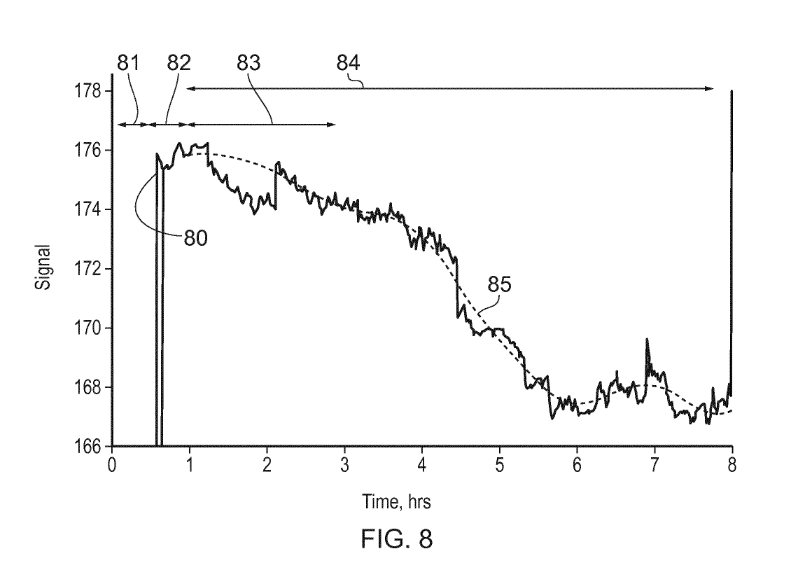

FIG. 8 shows a graph of the actual process trend data from FIG. 7 (curve 80), with a second order polynomial curve, being the calculated process trend data, shown also (dotted curve 85). The feed event stage t1 is indicated, as time 81, followed by the stabilisation period t2, having time 82. The measurement stage t3 follows, indicated as 84, and at the start of the measurement stage a shorter time period 83 is indicated; this is the minimum temporal window. Thus, the data is collected over the time period 83 and then used to fit the polynomial, and the fit continues with the actual process trend data past the end of the minimum temporal window to the end of t3, as indicated by the continued dotted curve 85 of the calculated process trend data.

Once the calculated process trend data is available via the curve fitting, it can be used in a determination of the protocol for feeding or other additive injections. The controller makes use of the calculated process trend to derive the protocol in a manner which is described further below, and then sends appropriate control signals to the pump(s) to deliver additive in accordance with the protocol.

In an embodiment, the calculated process trend is analysed by taking the coefficients of the polynomial, which may be for example a second order polynomial, and using them to calculate the derivative of the calculated process trend (e.g. the curve 85 in FIG. 8). The derivative of the calculated process trend may be referred to as the metabolic rate index (MRI), since it relates to the level of biological activity, or metabolism, of the bioreaction process. An advantage of this approach of fitting a low order polynomial to the actual process trend data and extracting a MRI as the derivative of this polynomial is that it is much less sensitive to noise than if derivatives of the simple measured signal are taken directly. This is because there are the previously discussed genuine fluctuations which represent real metabolic changes in the system and which are often greater than the noise, and observation of these fluctuations has been found to be of use in controlling bioreactions.

Now that the concept of the MRI has been defined, we return briefly to the minimum temporal window. Recalling that this window is effectively a buffer during which data is collected to a sufficient amount to ensure adequate accuracy of subsequent calculations, an example technique to determine with minimum temporal window duration is to find the length, or equivalently buffer size, that produces a minimum perturbation of the MRI value after that has been calculated. The derivative of the MRI is calculated and the buffer size/window length varied to find the value having the minimum peak-to-peak magnitude whilst maintaining the low residual of the curve fitting. This length is then used as the minimum temporal window length. In other words, the window length is determined retrospectively, by computational analysis of already-collected data over a range of buffer sizes to select that which has a minimum noise or error in the corresponding MRI value and also a good R² value for the fitted process trend (such as a value of 0.9 or more, for example). Other techniques may be used to determine a suitable window length or range of lengths deemed to give sufficient accuracy, however. A fixed window length can be set based on data from a calibration run, for example, or the window length may be dynamically updated according to current or recent or previous data, including using averaging over a selected number of previous feed cycles.

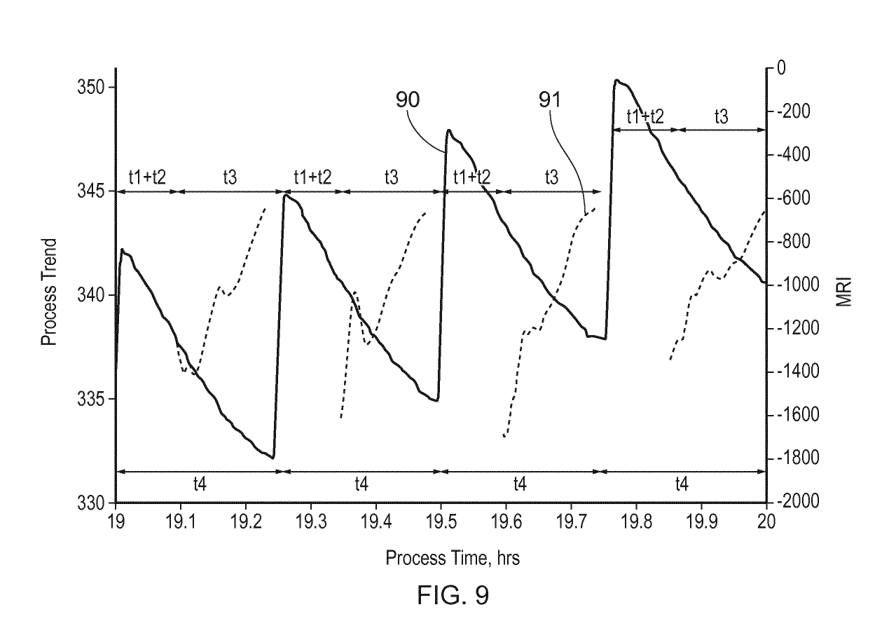

FIG. 9 shows a further graph of actual process trend data from later in a Saccharomyces cerevisiae fermentation run and including multiple feed cycles. The actual process trend (solid line 90, plotted on the left-hand vertical axis) is shown over four feed and consumption cycles (t4). The real time MRI values calculated for these cycles is shown as dotted lines 91, plotted on the right-hand vertical axis. Note the discontinuous nature of the MRI curves 91; this is because the MRI is derived from the calculated process trend data which is fitted to the actual process trend data 90 only over the t3 measurement time period and not the full t4 cycle time period, that is, not during t1 or t2.

The MRI can be used to determine feed conditions (additive protocol) in a number of ways. In a first example, the absolute value of the coefficients of the MRI may be used to determine feed events. In one example, the system monitors the MRI for a minimum absolute value based on prior knowledge of the general performance of the cell type in the bioreaction in question, and the minimum value triggers the next feed event. However, the absolute MRI value is prone to process-to-process variation, which while not being critical to method performance might cause a failure to properly control a feed event. Hence a more sophisticated approach may be preferred to obtain more reliable results. Under such an approach, the maximum MRI value that has occurred since the last additive injection (feed event) is identified and used to normalize the instantaneous MRI value. This normalised value can be thought of as a ratio. A threshold value may then be defined, and a trigger for a feed event (start of the next time period t1) is set to occur when the ratio drops below the threshold value. Useful values for the threshold are typically in the range from 0.1 to 0.9, and more usefully in the range 0.3 to 0.9, but will depend on the bioreaction in question. In example systems, a threshold value of 0.4 has been extensively employed. Hence, the time period t3 in the current t4 cycle will terminate at the next feed event, when t1 starts, and the length of t3 in subsequent cycles will likely vary in response to conditions in the reactor. Consequently, the invention provides for automatic delivery of a next feed event at the most appropriate time, giving excellent control over the bioreaction process. A ratio approach based on the normalised MRI value is useful in that it will adapt to cells that may be slow to start a bioreaction process but will recover if the feeding is adaptive in response to the cell performance. This adaptive response is not possible if the absolute MRI value is used for control. The ratio approach also compensates for any drift in the sensor performance, which can occur after prolonged immersion of the sensor in the bioreactor contents.

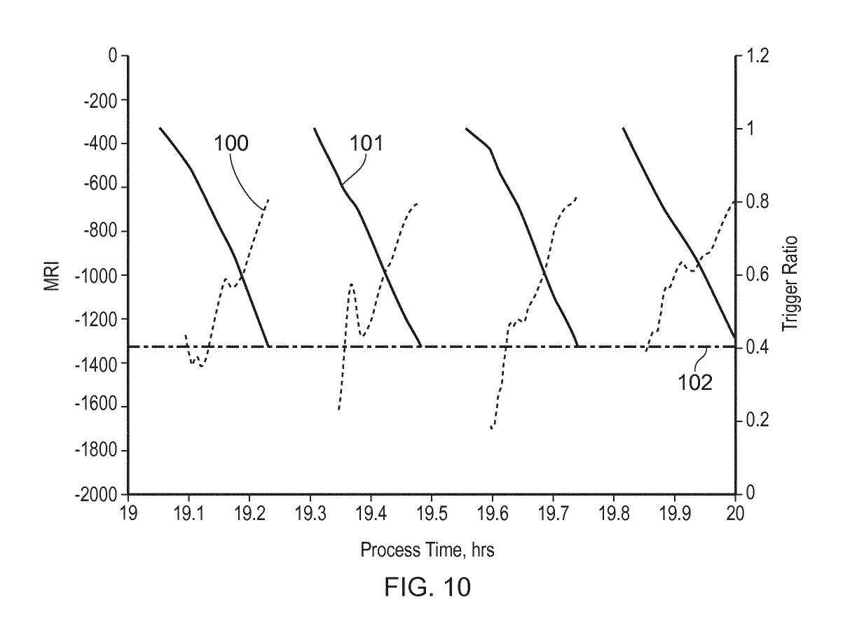

FIG. 10 shows a graph based on the same actual process trend data as FIG. 9. FIG. 10, however, shows the MRI curves as dotted lines 100, plotted on the left-hand axis, together with the corresponding trigger ratio (solid lines 101 plotted on the right-hand axis), being the ratio of the current MRI value divided by the absolute maximum MRI since the last feed pulse. In this example, the threshold for the trigger ratio was set at 0.4 (line 102) so that a feed event was triggered when the ratio fell below 0.4. Note again the discontinuous nature of the curves, arising from the fact that measurements of the bulk process property are disregarded during the feed event and the stabilisation time periods.

Hence, according to the embodiments of the invention, the controller is programmed to determine derivative values (the MRI) only during a fraction of the overall process; this enables more reliable control to be achieved. In particular, there is a time period immediately after instigating each feed event (designated t1) during which injection and mixing is occurring. There is a further, consecutive, time period (designated t2) during which the bioreactor contents is stabilising to return to an effective equilibrium state. Both of these time periods are ignored, and then during a subsequent time period (designated t3) derivative MRI information is calculated from the actual process trend data. The cycle repeats with a cycle duration t4=t1+t2+t3, but note that the length of t4 is altered to provide optimized feeding, by altering t3.

So, the invention proposes to calculate temporal differentials of a measured non-specific process signal, but to disregard the response that occurs during and immediately following each feed event. By choosing to calculate derivatives only in between feed events it is possible to correlate reactor activity sufficiently to determine a meaningful automated feeding protocol.

Thus far, the method has been described as comprising the fitting of a polynomial curve to the actual process trend data. The aim of this curve fitting is to obtain process trend data that corresponds to that which might be obtained from a heavily damped system, as this subsequently gives a MRI that more accurately represents the bulk changes in the bioreaction occurring in the bioreactor contents. Feed event predictions made using the MRI are thereby enhanced. It follows, therefore, that the invention is not limited to polynomial curve fitting; other mathematical methods that can similarly model a damped process can equivalently be used. Other mathematical curves may be fitted, for example an exponential curve, or an algebraic defined function, although other curves are not precluded. However, an exponential curve or a second order polynomial curve may be preferred; these have been found to give a highly satisfactory fit to typical process trend data and moreover are mathematical more simple to implement. Methods other than curve fitting may alternatively by used, for example the actual process data may be averaged, in particular heavily averaged to yield the calculated process trend data. Furthermore, in some systems it may be found that no mathematical processing of the actual process trend data is necessary at all, and the MRI can be obtained by directly calculating the derivative of the actual process trend data after t2 has expired. This may be applicable in very stable processes subject to little noise, for example.

Consequently, all descriptions herein referring to a fitted polynomial curve apply equally to process trend data treated by similar mathematical methods to model a damped system, and also to process trend data from which the MRI is directly derived without mathematical processing.

In a further embodiment the size of the feed delivered during a feed event may be varied to optimize the performance of the system and reduce the range of feed conditions (such as glucose concentration if the feedstock is glucose) to which the biological entity in the reaction is exposed. For example, the feed size may be set initially so that the requirement for a feed (indicated by the threshold for the ratio being passed) will not occur before the minimum temporal window condition is satisfied (in other words, t3≧minimum temporal window), and in addition so that the next feed event should occur after about twice the minimum temporal window size (t3≈2×minimum temporal window). It will be understood that other multiples of the temporal window size could be used for this initial set-up, from perhaps 1.2 to 10, although low multiples may be subject to unwanted effects from noise while higher ratios will have a correspondingly large feed volume with greater range of subsequent feed concentration within the reactor than may be desirable. Once the system is cycling through feed events (repeated execution of the t4 cycle) the feed size can be adjusted, perhaps to aim at a value of t3 having less extreme multiples of the minimum temporal window. An algorithm for determining feed size may also depend on information about previous feed size (for example the last feed or last three feed sizes), information on the maximum MRI during the previous feed cycle and information on the current MRI or process trend.

It will be understood that many control approaches using the MRI could be deployed as alternatives to those described above. The controller may be programmed with software configured to execute a desired algorithm for determine feed event times and feed sizes from the calculated MRI, and to send corresponding control signals to operate the pump(s) accordingly.

Thus far, this application has discussed feeding and additive protocols which are essentially on-off in nature, with zero feed during the measurement time and a discrete period of time during which feed is injected. In alternative embodiments, it is possible to employ a feed or additive rate that switches between two non-zero levels: a high level (in excess of the local feed consumption rate) and a low level (below the local feed consumption rate), thereby providing a continuous background feed at the low level. The period of time for which the high feed level is delivered corresponds to the feed event over time period t1, while the low feed level occupies the time periods t2 and t3. The high feed level and continuous background level (low feed level) can be selected to give a suitable signal-to-noise ratio for the MRI-derived control to operate effectively and can be modified throughout the process run as an optimized parameter. This method can also help reduce the variability of the feed level within a full run of a process, occupying many cycles.

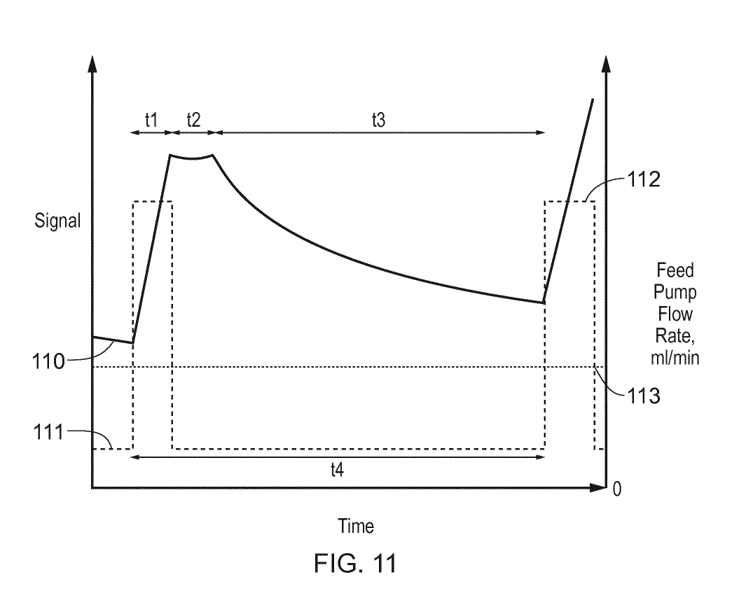

FIG. 11 shows an idealised example graph of events during a process having such a non-zero low-level baseline feeding approach. The feed cycle (over the time period t4) does not require a zero feed or additive flow during the t3 measurement phase. The measured signal 110 (for example, refractive index) is similar to that illustrated in FIG. 5. The pump delivering the feedstock or other additive into the bioreactor vessel has a flow rate (dashed line) that varies between two flow rates, a low rate 111 below the local feed consumption rate 113 and a high rate 112 during time period t1 greater than the local feed consumption rate 113. These values are constant during the relative phases (although the rate during the feed event period t1 may instead by varied), and as long as the flow rates stagger as shown to meet the local average feed requirement then the feeding calculation as already described remains the same. The impact of the non-zero feed rate during t3 will be a decrease in feeding frequency for any given feed volume, which may be a desired regime, for example to reduce pump usage, or to keep the reactor contents composition more uniform over time.

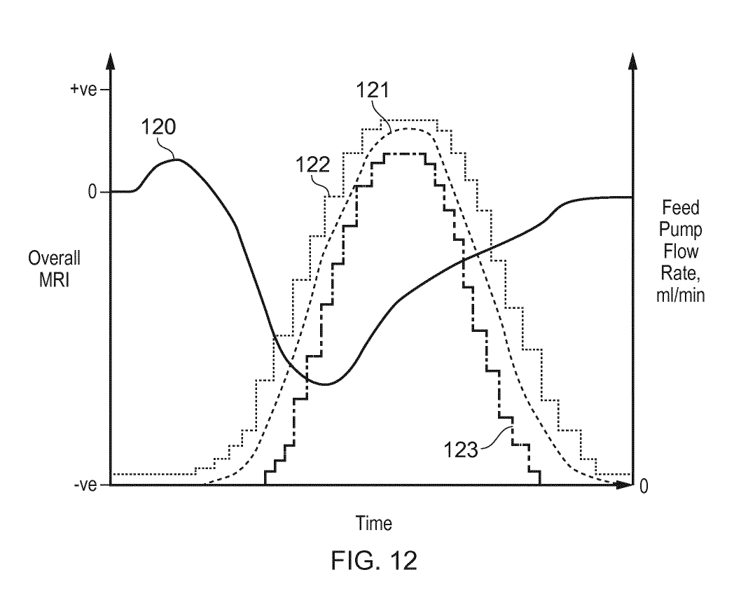

FIG. 12 shows an example graph illustrating a further non-zero baseline feeding approach, namely an adaptive feeding cycle that includes changing of both the low rate and high rate feed volumes during a process run of many cycles. The MRI 120 is plotted against the left-hand axis with the steps caused by separate feed events averaged out. This can represent the overall activity profile of a given process. The local feed consumption rate 121 is also shown, and this will vary over the process depending on the activity in the bioreactor at the time (note that this is a different parameter from both the process trend and the MRI, and is only obtained through the feeding phases). The previous example in FIG. 11 used fixed feed flow rates, but neither the high rate nor the low rate need to remain fixed. Rather, they can vary for each feed cycle, such as shown in FIG. 12. Based on the MRI value and length of the time to reach the trigger threshold for the previous cycle (duration of t3), the high flow rate and the low flow rate can be changed, perhaps in every cycle, as illustrated. These changing rates are shown respectively as lines 122 and 123, plotted against the right-hand axis. This will allow for the best conditions both for the organism and the measurement system to be maintained over the changing nature of the process.

The control method and system according to embodiments of the invention offer an advantage in that the MRI-triggered feeding protocol works regardless of the sign of the MRI value, and indeed, under the method the MRI may change sign during a process run without preventing the automated control from continuing to operate. A sign change may occur during a complex biological growth process, for example one in which an initial cell colony grows, multiplies, goes into production and then suffers cellular necrosis. Each of these changes may cause the underlying bulk physical properties of the bioreactor contents (such as refractive index) to be modified in complex ways (for example, during some stages the refractive index may be dominated by feed stuffs, and in stages by products of the process), yet embodiments of the invention can maintain control throughout the lifetime of the process.

In a further embodiment, the invention can be applied to a bioreaction involving multiple feedstocks or other additives, for example carried out in a system such as that shown in FIG. 2. The control method described thus far can be readily extended to the control and delivery optimization for multiple feedstocks.

This can be implemented, in some embodiments, by independently controlling the injections of each feedstock or additive according to a predefined pattern or sequence, analysing the derivative data (that is, the MRI) resulting from each injection (feed event), and then modifying the feeding protocol as indicated by the analysis, for example by switching to a different feedstock if a feed event is deemed a failure.

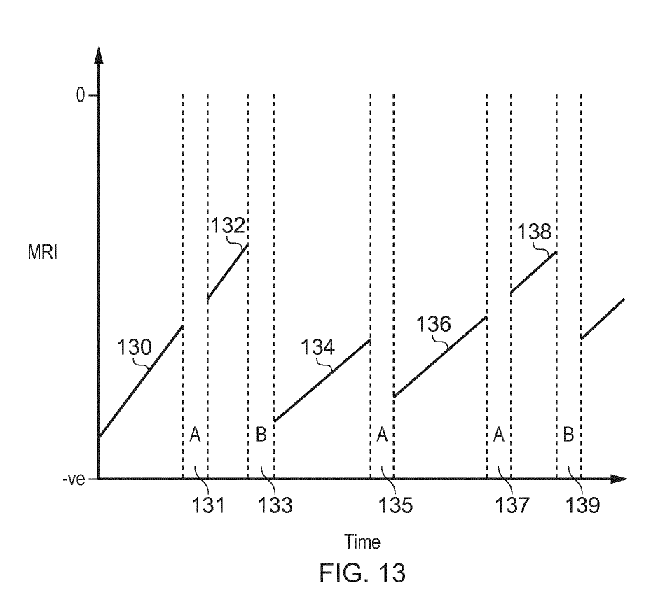

FIG. 13 shows a graph with plots of idealised data representing control of a reaction having multiple feedstocks. The MRI 130 for an example process varies from a value with a large absolute value (plotted as a negative value in the Figure) to a lower value during a first depicted period. Once MRI has dropped below the predetermined trigger threshold, a feed event is triggered for a primary feed, such as Feed A. After the feed event time t1 and the following stabilisation period t2 (131) have elapsed, and the measurement period t3 is entered, in this example it becomes apparent that the MRI value 132 has continued from the previous value 130 and has not responded to the Feed A feed event (within an allowable tolerance, for example). Once the minimum temporal window has passed for a reliable measurement (so that the calculated MRI is reasonably certain), a feed event for a second feed, Feed B can be triggered. This is delivered during t1, with a stabilisation period t2 (133). An increase in the MRI 134 in the subsequent measurement period is observed, and the feed event is deemed successful. However, if the feed event for Feed B had not produced in an increase in the MRI, it would be deemed unsuccessful, and a feed event for third feed, Feed C, can be triggered, in the same manner as Feed B was used to replace Feed A in the previous cycle. Alternatively, an operator may be automatically notified. In the present successful case, though, the process continues with the next feed event reverting to Feed A (135), which produces an increase in the MRI 136, although more slowly than previously. A further feed event using Feed A (137) again fails to increase the MRI 138, so that it is replaced by Feed B in the next feed event (139), applied after the minimum temporal window as before. The cycle is continued until the process reaches the required end point and is terminated.

To summarise, in an example use of multiple feedstocks, a successful feed event increases the MRI and is followed by a feed event with the primary feedstock after the full measurement time, whereas an unsuccessful feed does not increase the MRI so is followed by a feed event with a different feedstock after a measurement time limited to the minimum temporal window, until all available feedstocks have been tried. An appropriate next stage will then be triggered, for example, user intervention obtained via an automatic alert, or a return to the primary feedstock.

Additionally, the system may operate in a more complex multiple feedstock mode in which the number of effectual (successful) and ineffectual (unsuccessful) feeds of each feedstock are tracked within the process. In response to an ineffectual feed, the corresponding feedstock is moved to a lower index or order in the feed sequence. So, in terms of the above example, the A-B-C feedstock sequence is not fixed, but rather is dynamically varied over the process run. Also, the total dose of each feedstock can be logged against time and then used to determine a “seed” or initial feedstock sequence for subsequent runs of the same process, such that the highest dose feedstock is set as the future primary feedstock, for example. This dose information may also usefully be used to infer feed composition and dose rates for non-feedback controlled runs of the same cell line process.

As an additional modification, it is possible that the controller be programmed to add feedstocks or other additives which are not controlled by the described MRI feedback loop. For example, one or more additional additives may be injected on a periodic feed cycle at a fixed rate independent of the feed event times determined by the feedback control. In another example, the controller may make injections on a pre-set cycle in addition to the feedback control, so that if a given feed has not been required by the control algorithm for greater than a set time (perhaps 24 hours) then a small dose of that feedstock could be added automatically or on request by an operator. Additionally, the controller may be configured to treat any feed event of these or other types (such as a manual feed injection by an operator) as a regular feed event in the t4 cycle so that the duration of this feed event is treated as t1 and taken as the commencement of the next t4 cycle used to set the time for the next automatic feed event. This allows the addition of a component, additive or feedstock outside of the controlled process to be added at the discretion of a user, perhaps as a “one-off” event, without disrupting the automated process control provided by the controller.

Embodiments of the invention may further be used to determine the end-point of a process. This can be achieved by analysis of the MRI value to determine the point at which the absolute MRI value (rather than the trigger ratio value discussed already) drops below some predetermined threshold value, considered to mark an end-point condition. It will be understood that other approaches may be adopted, for example if the time between feed events (length of t4) extends beyond some maximum value, or if the process trend (absolute or calculated) moves beyond certain predetermined control values. The controller can be configured to monitor for one or more of these criteria, and take appropriate action in response to a criterion being fulfilled, such as sending a warning to operators that the end-point is approaching. This ability to automatically determine a process end-point can be useful in an industrial setting as it allows for improved use of capital infrastructure.

Similarly, the controller can monitor for other events that have a measurable effect on the bioreactor contents bulk properties and/or the MRI, and send information to operators warning them of certain conditions such as the failure of a feed to occur properly, or contamination/infection of the bioreactor media. Feed failure can include a range of “fail” events, including practical failures such as a broken pump or empty reservoir meaning that no feedstock is delivered, and failures within the process whereby a feed event is deemed ineffectual or unsuccessful in that addition of feed does not produce the expected change in the MRI.

The automation described thus far enables a bioreactor system to be a stand-alone system that does not require user contact, and may further be to configured to automatically transition the product to the next processing stage after the end-point is detected, for example heat termination, or draining the reactor vessel. However, by using communications (including wireless communication and the Internet) it is possible for the system to alert operators to the process status. The alert may be in the form of warning displays, text messages to mobile phones, audible warnings and the like, all produced by the controller in response to certain detected conditions. The controller may also log and escalate responses. For example, if an event occurs which is pre-determined to require a user input and that input does not occur within a defined time, then a super-user or higher level supervisor may be contacted automatically. Such a situation might occur if a feedstock runs out, a pump fails, a pipe leaks, a connector breaks, or an operator steps on or kinks a pipe. The controller may be programmed to diagnose these and other failures to provide a feed event (which will be apparent when there is no discernible change in the process trend following transmission of a feed injection control signal) and warn appropriately.

As mentioned with reference to FIG. 1, the bioreactor system may include feedback loops for control of temperature and other conditions. The feeding control described thus far can be implemented entirely independently of these other controls. Alternatively, the MRI calculated for the feeding control implementation may also be used to control other conditions, such as the operating temperature. Hence, the controller may be configured to analyse the MRI data, and use it to generate further control signals which are sent to other devices in the system, such as the heater. Also, the controller can receive input signals from other sensors, such as temperature and pH sensors and generate control signals based on these measurements, so that most or all of the bioreactor system has an operating regime automated by a single controller.

As mentioned above, the system can be configured so that the controller sends an alarm or warning signal if an ineffectual feed event occurs. A supplementary arrangement may be provided which instigates a back-up feed if an alarm state is entered. The back-up feed may be provided by a back-up additive supply mechanism configured to deliver one or more feedstocks for the current process, and which is activated by a control signal from the controller around the time that the controller sends the alarm signal. The feedstock from the back-up mechanism is hence delivered into the bioreactor to replace the ineffectual feed event, so that the process is able to continue even if the primary additive supply mechanism fails for some reason to provide an effectual feed. The product of the process is hence not lost.

As a further embodiment, the back-up additive supply mechanism can be under the control of a secondary microprocessor, independent of the microprocessor discussed so far (component 12a in FIG. 1, for example) but configured and connected in the same way. A failure of the main microprocessor will both transmit the alarm or warning signal, and alert and activate the secondary microprocessor which assumes control of the system and produces the next feed event or events. The secondary microprocessor might therefore have control of the back-up additive supply mechanism and/or the main additive supply mechanism and send feed control signals thereto as appropriate, and might have an emergency power supply in case the failure of the main microprocessor is caused by a power failure.

In summary, the invention relates to methods and systems for automating the control of the feeding of a bioreactor. In an embodiment, a system comprises an in situ measuring sensor device (for example one responding to refractive index), a controlled feeding mechanism (such as a pump and reservoir of nutrient), and a control box containing a processing unit that automatically analyses the response of the cells to the injection of the nutrient in such a way as to allow control of the bioprocess. The injection may uses pulses or may involve a continuously varying injection amount. The control system analyses the magnitude and timing of the response of the sensor to the injection stimulus. The decision on feeding may be made based on the control system and pre-programmed information about the anticipated response to a feed event.

### REFERENCES

[1] F. Lu, P. C. Toh, I. Burnett, F. Li, T. Hudson, Automated Dynamic Fed-Batch Process and Media Optimization for High Productivity Cell Culture Process Development, Biotechnology and Bioengineering, 110, 1, 191-205 (2013).
[2] Akesson, M., Hagander, P., Axelsson, J. P., A probing feeding strategy for Escherichia coli cultures, Biotechnology Techniques, 13, 523-528 (1999).
[3] Akesson, M., Hagander, P., Axelsson, J. P., Probing control of fed-batch cultivations: analysis and tuning. Control Engineering Practice, 9, 709-723 (2001).
[4] PCT/GB2005/002680
[5] PCT/GB2005/002682

1. A method of controlling additive delivery during a bioreaction in a bioreactor, the method comprising:
running a bioreaction in a bioreactor including adding additive into the bioreactor during spaced-apart feed events, where contents of the bioreactor equilibrate during a stabilisation period after a feed event;
making in situ measurements of a bulk physical property of the bioreactor contents during the bioreaction to obtain process trend data;
calculating a derivative of process trend data obtained over a measurement period beginning after a stabilisation period, the derivative being a metabolic rate index (MRI); and
using the MRI to determine a time for starting a next feed event.

2. A method according to claim 1, in which the process trend data obtained via the in situ measurements is actual process trend data, the method further comprises applying a mathematical method to actual process trend data obtained over a measurement period beginning after a stabilisation period to produce calculated process trend data representing a damped version of the actual process trend data, and the calculating a derivative comprises calculating a derivative of the calculated process trend data.

3. A method according to claim 2, in which the mathematical method comprises an averaging of the actual process trend data, or a fitting of a mathematical curve to the actual process trend data, or a fitting of a second order polynomial curve to the actual process trend data.

4. (canceled)

5. A method according to claim 1, in which the bulk physical property is refractive index.

6. A method according to claim 5, in which making in situ measurements of refractive index comprises using a sensor configured to detect changes in a propagating evanescent wave.

7. A method according to claim 6, in which the sensor detects changes in a modal index.

8. A method according to claim 7, in which the sensor is based on a Bragg grating.

9. A method according to claim 1, in which the bulk physical property is density, conductivity, inductance, impedance, viscosity, turbidity, or spectral absorption at a single wavelength.

10. A method according to claim 1, comprising using the MRI directly to determine a time for starting the next feed event.

11. A method according to claim 1, in which using the MRI to determine a time for starting the next feed event comprises calculating a ratio by dividing the instantaneous MRI value by an absolute maximum value of the MRI since the previous feed event, comparing the ratio to a threshold value, and starting the next feed event when the ratio passes the threshold value.

12. A method according to claim 11, in which the threshold value is in the range of 0.3 to 0.9.

13. A method according to claim 1, in which the measurement period commences with a minimum temporal window, and the applying of the mathematical method and calculating of the derivative begins on process trend data collected during the minimum temporal window after the minimum temporal window expires.

14. A method according to claim 13, in which the minimum temporal window has a duration such that sufficient process trend data is collected for the corresponding MRI to have a minimum error or noise value compared to that for other durations.

15. A method according to claim 13, in which the duration of the minimum temporal window is adjusted based on previous process trend data.

16. A method according to claim 1, in which the stabilisation period has a duration determined by observation of process trend data obtained during a calibration run of the bioreactor.

17. A method according to claim 1, further comprising applying noise filtering to the obtained process trend data.

18. A method according to claim 1, further comprising varying the amount of additive delivered in a feed event in response to the MRI.

19. (canceled)

20. A method according to claim 1, in which additive is added into the bioreactor at a first rate during each feed event, and additive is added continuously into the bioreactor between feed events at a second rate lower than the first rate.

21. A method according to claim 20, in which one or both of the first rate and the second rate are varied over time.

22. A method according to claim 1, in which different additives are added into the bioreactor during different feed events, the MRI during a current measurement time being used to determine the additive for the next feed event.

23. A method according to claim 1, in which the or each additive is a direct or indirect feedstock.

24. A method according to claim 1, further comprising using the MRI to determine values for one or more operating conditions of the bioreactor.

25. A controller for controlling additive delivery during a bioreaction in a bioreactor, the control configured to:
receive in situ measurements of a bulk physical property of contents of a bioreactor during a bioreaction, the bioreaction including additive being delivered into the bioreactor during spaced-apart feed events, where contents of the bioreactor equilibrate during a stabilisation period after a feed event, the received measurements being process trend data;
calculate a derivative of process trend data obtained over a measurement period beginning after a stabilisation period, the derivative being a metabolic rate index (MRI);
use the MRI to determine a time for starting a next feed event; and
generate a control signal configured to cause an additive supply mechanism to deliver additive into the bioreactor at the determined time for the next feed event.

26-42. (canceled)

43. A system for controlling additive delivery during a bioreaction in a bioreactor, the system comprising:
a bioreactor
an additive supply mechanism configured to deliver additive into the bioreactor during feed events, in response to a control signal;
a sensor associated with the bioreactor and configured to make in situ measurements of a bulk physical property of the bioreactor contents during a bioreaction, thereby obtaining process trend data; and
a controller configured to:
receive process trend data from the sensor;
calculate a derivative of process trend data obtained over a measurement period beginning after a stabilisation period during which contents of the bioreactor equilibrate after a feed event, the derivative being a metabolic rate index MRI;
use the MRI to determine a time for starting a next feed event; and
send a control signal to the additive supply mechanism to deliver additive at the determined time for the next feed event.

44-65. (canceled)
